(19) United States
(12) Patent Application Publication
(10) Pub. No.: US 2015/0150708 A1
(43) Pub. Date: Jun. 4, 2015

(54) LOCKABLE HINGE

(71) Applicant: Camp Scandinavia AB, Helsingborg (SE)

(72) Inventor: Juan Bautista Paez, Rockaway, NJ (US)

(21) Appl. No.: 14/547,109

(22) Filed: Nov. 18, 2014

Related U.S. Application Data
(60) Provisional application No. 61/910,247, filed on Nov. 29, 2013.

(51) Int. Cl. A61F 5/01

(52) U.S. Cl. CPC A61F 5/0123; A61F 2005/0158; A61F 2002/6854

(57) ABSTRACT

The invention relates to a lockable hinge for an orthosis. The lockable hinge comprises a first side bar and a second side bar, a bridging element and a locking mechanism. A first end of the first side bar is fixedly mounted to a first portion of the bridging element, and a first end of the second side bar is rotatably mounted to a second portion of the bridging element. The locking mechanism is supported by the first side bar, and the locking mechanism comprises a locking block being slidable along the longitudinal extension of the first side bar between a locked position and an unlocked position. In the locked position the locking block engages by wedging between the second side bar and the bridging element, whereby the second side bar in the locked position is fixedly aligned with the first side bar.

### CROSS-REFERENCE TO RELATED APPLICATIONS

This application claims the benefit of U.S. Provisional Application No. 61/910,247, filed Nov. 29, 2013, the entire contents of which is hereby incorporated by reference.

### TECHNICAL FIELD

The present invention relates to a lockable hinge for an orthosis and an orthosis using such lockable hinge.

### BACKGROUND

Orthoses are used to support and assist/control different joints of the body. Some of them, such as those for knees and elbows may be provided with a lockable hinge. The lockable hinge allows a well controlled rest position of the body joint, while still allowing a well controlled bending movement when required. Typical such occasions are physiotherapy, dressing or changing position from a standing position to a sitting position.

For support/assist of the knee joint it is well known to use a so called knee ankle foot orthosis, also known as a KAFO. A KAFO is a device intended for persons with a weakness in the muscles and ligaments that controls the knee. A

KAFO is most commonly used by persons with knee extension deficits, isolated issues or in combination with other knee control problems. The typical diagnosis for a user of a KAFO is post-polio syndrome, multiple sclerosis, spinal trauma or muscle dystrophy.

A typical KAFO comprises supports on opposite sides of the knee, the supports extending from the thigh to the calf. To allow bending of the knee the supports are provided with a hinge at the knee level. The hinge on one or both sides of the knee is arranged to be locked when the user is standing or walking, but has to be releasable to allow the knee to bend when the patient is e.g. sitting or is doing physiotherapy. To allow such release it is well known in the art to use a locking mechanism in the form of a ring catch or a drop lock function.

One example of a KAFO with a drop lock function is found in U.S. Pat. No. 2,591,373. The KAFO comprises an upper and a lower brace member pivotally connected by a hinge pin. The lower end of the upper brace is provided with a mating segmental arcuate surface so that when the brace is in straight extended position, the weight of the user is directed through the upper brace member and the abutting surfaces to the lower brace member. In order to regulate and limit the pivoting motion of the upper brace member about the hinge pin, the lower brace member is provided with a radially recessed arcuate surface while the upper brace member is provided with a locking lug which projects downwardly against the arcuate surface to thereby form a limit stop. A spring biased locking block is provided. To allow bending of the knee, the locking block is retracted by the user by operating a lock operating finger extending along the upper brace member.

### SUMMARY OF THE INVENTION

It is an object of the present invention to provide a lockable hinge for an orthosis. The lockable hinge should be applicable to any orthosis using a lockable hinge.

The lockable hinge should be provided with a built in safety, i.e. if the operation means of the lockable hinge fails, the hinge should go into a locking position.

The lockable hinge should allow an easy and simple adjustment.

The lockable hinge should be able to withstand the high forces and the wear that a lockable knee hinge will be subjected to during its lifetime.

Further, the lockable hinge should provide a high degree of stability for the user to feel safe and additionally, the lockable hinge should have a slim, rigid and robust design.

These and other objects are met by a lockable hinge for an orthosis, the lockable hinge comprising a first side bar and a second side bar, a bridging element and a locking mechanism, wherein a first end of the first side bar is fixedly mounted to a first portion of the bridging element, and a first end of the second side bar is rotatably mounted to a second portion of the bridging element, and the locking mechanism is supported by the first side bar, and the locking mechanism comprises a locking block being slidable along the longitudinal extension of the first side bar between a locked position and an unlocked position, in which locked position the locking block by wedging engages between the second side bar and the bridging element, whereby the second side bar in the locked position is fixedly aligned with the first side bar.

By the wedging engagement, a lockable hinge is provided which by the user will be experienced as very rigid without any play. Also, by the wedging engagement the locking function will be maintained although the lockable hinge should be subjected to wear.

By the firsts ends of the first and second side bars being mounted to different portions of the bridging element and thus to different connecting points, instead of being located to one and the same connecting point, a more favourable load distribution is provided. This allows for a prolonged lifetime.

The principle is applicable no matter if it is applied to an orthosis to be used for an elbow or a knee and also no matter the complexity of the orthosis, e.g. whether it is a plain knee orthosis or a complex KAFO.

The bridging element may comprise a first flange overlapping a portion of the long side of the first end of the first side bar and a portion of the long side of the first end of the second side bar; and a bridging portion overlapping at least a portion of the short side of the first end of the first side bar and a portion of the short side of the first end of the second side bar, the bridging portion extending essentially perpendicular to the first flange, whereby the locking block is arranged to engage by wedging between the second side bar and an inner surface of the bridging portion.

The bridging element may further comprise a second flange, opposite the first flange and connected thereto via the bridging portion, whereby the second flange is overlapping a portion of the opposite long side of the first end of the first side bar and a portion of the opposite long side of the first end of the second side bar.

The thus resulting wedging engagement surface will be protected from at least two sides, i.e. by the first flange and the bridging portion of the bridging element and by the second flange if any. This protects the locking mechanism from being contaminated with dirt, such as textile fibers. Also, any clothing, blankets or the like is prevented from being getting stuck in the hinge.

The lockable hinge may further comprise a first distance element comprising a flange and a shoulder, the shoulder extending along the flange, wherein the flange overlaps a portion of the long side of the first end of the first side bar, and the shoulder having an inner surface overlapping a portion of the short side of the first end of the first side bar, and an outer surface complementary to and in contact with an inner surface of the bridging portion of the bridging element.

By the distance element the first side bar may be formed by an off-the-shelf piece with a rectangular cross section. The distance element serves the purpose of filling up the play between the flange/flanges of the bridging element. Also the shoulder contributes to the formation of a sliding surface for the locking block. Also, it contributes to the definition of a locking cavity guiding the locking block when being moved between its locked and unlocked position.

The thickness of the first side bar and the thickness of the flange of the first distance element may correspond to the distance between the first and the second opposing flanges of the bridging element. Thereby there will be no play between the first side bar and the bridging element that may be experienced as an instability by the user.

The shoulder may comprise a through channel extending along the longitudinal extension of the shoulder, and wherein the through channel may receive a wire which in one end is connected to the locking block, whereby the sliding movement of the locking block may be operated by the wire being pulled. By the wire at least partly being received in a through channel, dirt such as textile fibers is prevented from entering and interfering with the locking mechanism. Also, the wire may easily be directed away from the lockable hinge towards and along the first side bar.

The locking block may have an upper guiding surface complementary to the inner surface of the bridging portion of the bridging element. By the complementary geometry, any play there between is eliminated and thereby the risk of the user experiencing any instability is reduced.

The locking block may be biased towards the locked position, whereby the lockable hinge is provided with a built in safety, i.e. if the operation means of the lockable hinge fails, the hinge should go into a locking position.

The locking block may have a longitudinal extension bridging the first ends of the first and the second side bars and thereby always be in contact with both side bars. This provides a rigid operation and a robust and stable experience by the user.

The locking block may be slidably received in a locking cavity defined by the inner wall of the bridging portion and at least one of the flanges of the bridging element and the short sides of the first ends of the first and second side bars. The walls of said locking cavity provides a guiding of the locking block during its movement between the locked and unlocked positions.

The locking block may be operable via a remote control. The remote control allows an easy operation of the lockable hinge without the user requiring any tooling. Also, the lockable hinge may be operated by the user even if he/she should have a reduced mobility. The remote control may be placed at a position allowing an easy access, such as along the thigh under a skirt or on the waist belt or the like.

According to another aspect, the invention may relate to a knee ankle foot orthosis, a knee orthosis or an elbow orthosis comprising a lockable hinge with the features given above.

It is noted that the invention relates to all possible combinations of features recited in the claims.

### BRIEF DESCRIPTION OF THE DRAWINGS

This and other aspects of the present invention will now be described in more detail, with reference to the appended drawings showing one embodiment of the invention. Like reference numerals refer to like elements throughout the drawings.

FIG. 1 illustrates a general overview of the components of the lockable hinge according to the invention.

FIG. 2a illustrates the lockable hinge according in its locked position.

FIG. 2b illustrates the lockable hinge in its unlocked and bent position.

FIG. 3 illustrates one embodiment of the first distance element.

FIG. 4 illustrates one embodiment of the second distance element.

FIG. 5 illustrates one embodiment of the bridging element.

FIGS. 6a and 6b illustrates the lockable hinge applied to one embodiment of a knee ankle foot orthosis.

### DETAILED DESCRIPTION

The present invention will now be described more fully hereinafter with reference to the accompanying drawings, in which currently preferred embodiments of the invention are shown. The invention may, however, be embodied in many different forms and should not be construed as limited to the embodiments set forth herein. Rather, these embodiments are provided for thoroughness and completeness, and fully convey the scope of the invention to the skilled person.

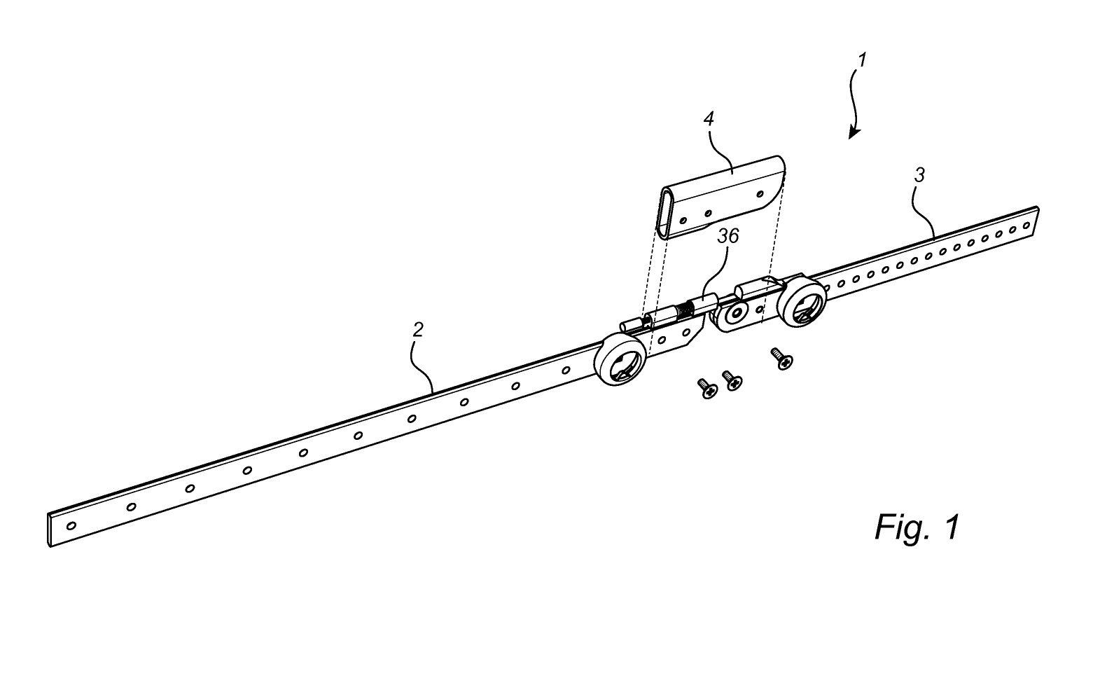

Referring to FIG. 1, a general overview of the lockable hinge 1 is disclosed. The lockable hinge 1 comprises in its most simple form a first side bar 2 and a second side bar 3 interconnected by a bridging element 4. The lockable hinge further comprises a locking mechanism comprising a slidable locking block 36.

For ease of understanding, in the following description, the first side bar 2 is intended to extend along the thigh of the user, whereas the second side bar 3 is intended to extend along the calf of the user. Still, it should be stressed that he lockable hinge is applicable also to other body joints such as the elbow.

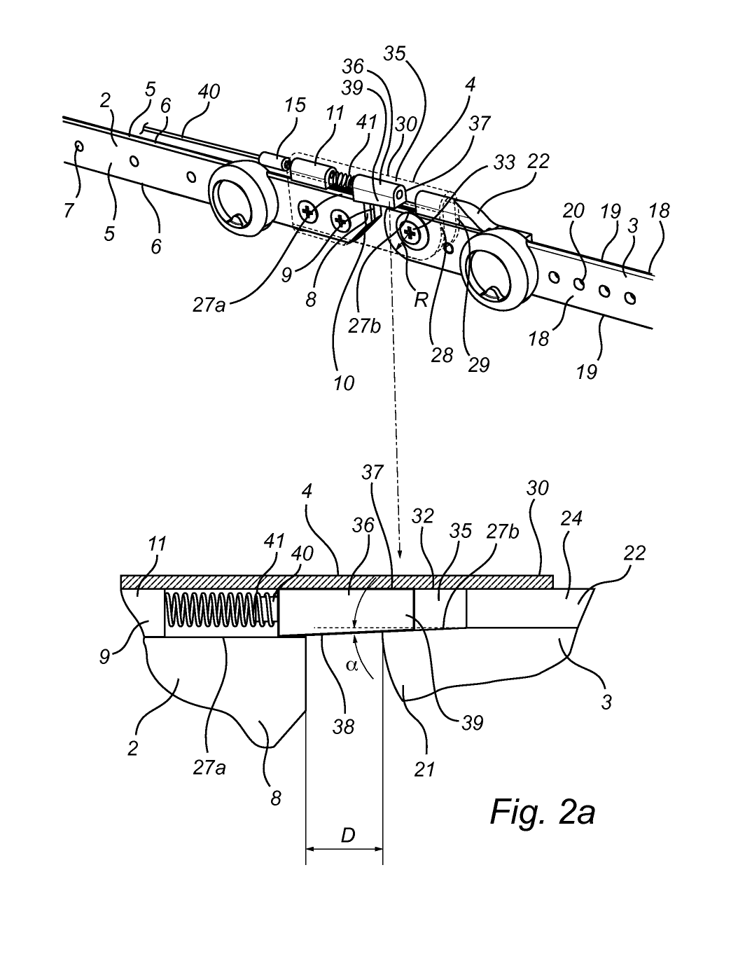

Referring to FIG. 2a, the first side bar 2 has a longitudinal extension having a rectangular cross section. Thus, the first side bar 2 has two opposing long sides 5 interconnected by two opposing short sides 6, with one of the long sides 5 intended to extend essentially facing the thigh of the user. The first side bar 2 may be made of metal, plastics, composite material or a combination of these materials.

In the disclosed embodiment the first side bar 2 has a number of holes 7 along it longitudinal extension. The holes 7 may be used to fix the first side bar 2 to an attachment around the thigh. The first side bar 2 may be provided in a fixed length to allow customizing depending on the size of the user.

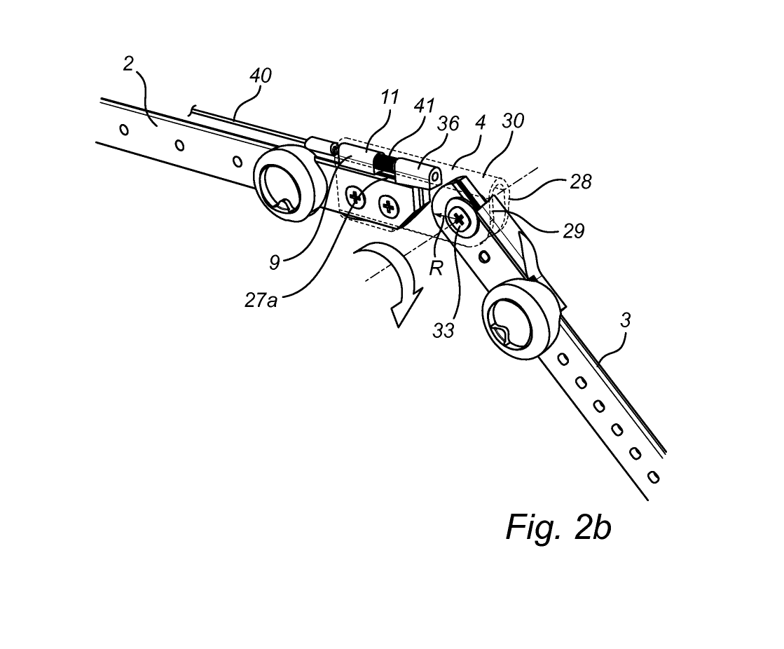

The first side bar 2 has a first end 8 oriented to face the second side bar 3. The first end 8 is fixedly connected to a first distance element 9, see FIG. 2a and FIG. 3. In FIG. 3, the first distance element 9 is schematically disclosed together with the first side bar 2, the first side bar being shown with dashed lines. The fixed connection may be provided by screws, rivets, adhesive or the like. It is to be understood that the first distance element 9 may be fixedly connected to the first side bar 2 by being integrated therewith, whereby no mounting is required.

The first distance element 9 comprises a flange 10 and a shoulder 11. The shoulder 11 extends essentially perpendicular to and longitudinally along the flange 10. The longitudinal extension of the flange 10 exceeds the longitudinal extension of the shoulder 11. When mounted to the first end 8 of the first side bar 2, the flange 10 overlaps a portion of the long side 5 of the first end 8 of the first side bar 2.

The shoulder 11 has an inner surface 12 overlapping a portion of the short side 6 of the first end 8 of the first side bar 2. Further the shoulder 11 has an outer surface 13 facing away from the inner surface 12. In the disclosed embodiment the outer surface 13 has a convex cross sectional profile although it is to be understood that other profiles are possible. The profile of the outer surface 13 is complementary to a guiding surface 14 of the bridging element 4 to be described below.

The shoulder 11 has a thickness T3 corresponding to the sum of the thickness T1 of the first side bar 2 and the thickness T2 of the flange 10 of the first distance element 9. When the first distance element 9 is connected to the first side bar 2, the inner surface 12 of the shoulder 11 will rest against the short side 6 of the first side bar 2 and be in level with the same.

The shoulder 11 comprises a through channel 15 extending along the longitudinal extension of the first side bar 2. The longitudinal centre axis C of the through channel 15 coincides with the parting line P between the first side bar 2 and the flange 10.

The width W1 of the flange 10 of the first distance element 9 corresponds to the width W2 of the first side bar 2. Thereby the short side 6 of the first side bar 2 facing the shoulder 11 will be in level with the inner surface 12 of the shoulder 11 thereby forming a sliding surface 27a to be discussed below.

The first distance element 9 may be made of metal, plastics, composites or a combination of these materials.

Now turning to FIG. 2a, the second side bar 3 has a longitudinal extension having a rectangular cross section. Thus, the second side bar 3 has two opposing long sides 18 interconnected by two opposing short sides 19, with one of the long sides intended to face the lower leg of the user. The second side bar 3 may be made of metal, plastics, composite material or a combination of these materials.

In the disclosed embodiment the second side bar 3 has a number of holes 20 along it longitudinal extension. The holes 20 may be used to fix the second side bar 3 to an attachment around the calf. The second side bar may be provided in a fixed length to allow customizing depending on the size of the user and its needs.

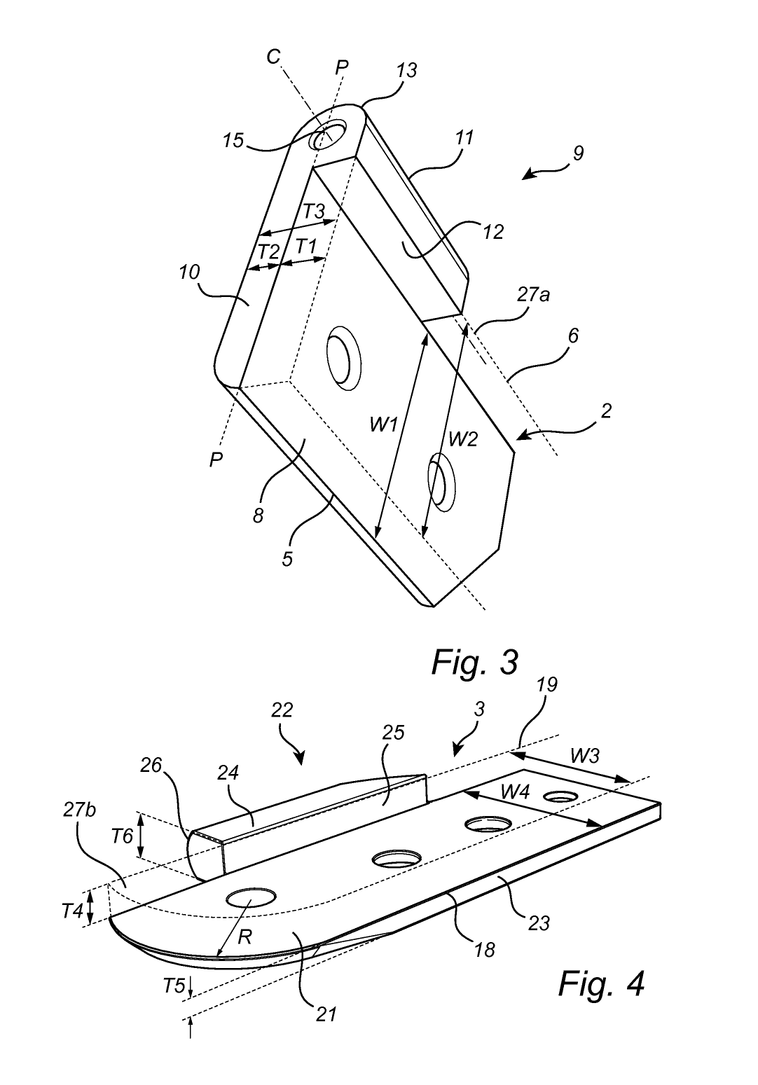

The second side bar 3 has a first end 21 oriented to face the first end 8 of the first side bar 2. The first end 21 is fixedly connected to a second distance element 22, see FIGS. 2a and 4. The fixed connection may be provided by screws, rivets, adhesive or the like.

It is to be understood that the second distance element 22 may be fixedly connected to the second side bar 3 by being integrated therewith, whereby no mounting is required.

The second distance element 22, see FIGS. 2a and 4, comprises a flange 23 and a shoulder 24. The shoulder 24 extends essentially perpendicular to the flange 23. The longitudinal extension of the flange 23 exceeds the longitudinal extension of the shoulder 24. The flange 23 overlaps a portion of the long side 18 of the first end 21 of the second side bar 3. The shoulder 24 has an inner surface 25 overlapping a portion of the short side 19 of the first end 21 of the second side bar 3. Further the shoulder 24 has an outer surface 26 facing away from the short side 19 of the second side bar 3. In the disclosed embodiment the outer surface 26 has a convex cross sectional profile although it is to be understood that other profiles are possible. The profile of the outer surface 26 is complementary to the guiding surface 14 of the bridging element 4 to be described below.

The shoulder 24 has a thickness T6 corresponding to the thickness T4 of the second side bar 3 and the thickness T5 of the flange 23 of the second distance element 22. Thus, when the second distance element 22 is connected to the second side bar 3, the inner surface 25 of the shoulder 24 will rest against the short side 19 of the second side bar 3 and being in level with the same. Further, the width W3 of the flange 23 of the second distance element 22 corresponds to the width W4 of the second side bar 3. Thereby the short side 19 of the second side bar 3 facing the shoulder 24 will be in level with the inner surface 25 of the shoulder 24 thereby forming a sliding surface 27b to be discussed below.

The second distance element 22 may be made of metal, plastics, composites or a combination of these materials.

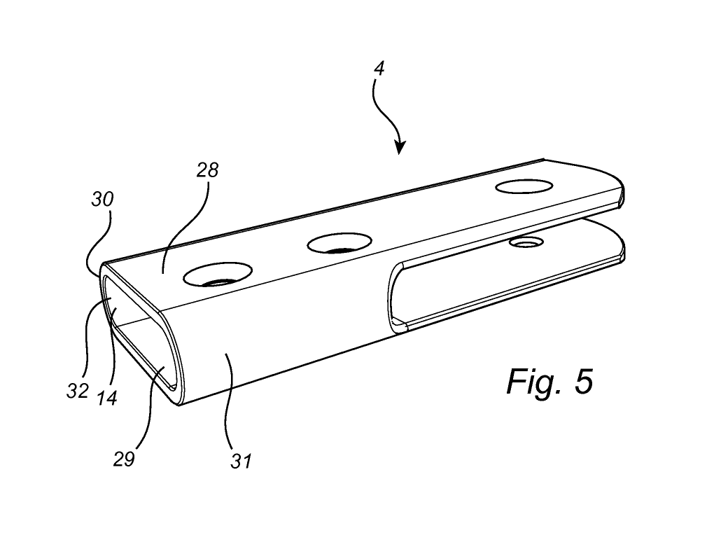

Now turning to FIGS. 2a, 3 and 5, the bridging element 4 is arranged to extend along the longitudinal extension of the first and the second side bars 2, 3 and connect to the first and the second side bars when connected thereto. A distance D is formed between the two first ends 8, 21 thereof.

The bridging element 4 comprises a first flange 28 and a second flange 29 interconnected by a bridging portion 30. The first and the second flanges 28, 29 are essentially parallel and separated by a distance corresponding to the total thickness T3 of the first side bar 2 and the flange 10 of the first distance element 9.

The bridging portion 30 is provided with a profile having an inner surface 32 complementary to the outer surface 13 of the shoulder 11 of the first distance element 9 and the outer surface 26 of the shoulder 24 of the second distance element 22. The bridging element 4 may be made of metal, plastics, composites or a combination of these materials.

In the disclosed embodiment, the bridging element 4 comprises an optional additional bridging portion 31. The additional bridging portion 31, being arranged opposite the first bridging portion 30 does not extend along the full length of the first and the second flanges 28, 29 but only along the portion thereof being arranged to overlap the first end 8 of the first side bar 2.

In its mounted position, the bridging portion 30 is arranged to straddle the short sides 6, 19 of the first and the second side bars 2, 3. Further the two flanges 28, 29 of the bridging element 4 are arranged to overlap a portion of the first ends 8, 21 of the first and the second side bars 2, 3 respectively with a longitudinal distance D between the first ends 8, 21 respectively. Thus there is no contact between the first ends 8, 21 of the first and second side bars 2, 3.

The first side bar 2 is fixedly mounted to the bridging element 4 by the bridging element 4 straddling the first end 8 of the first side bar 2 and the flange 10 of the first distance element 9. The first and second flanges 28, 29 of the bridging element 4 are arranged to overlap a portion of the long side of the first side bar 2. The overlapping area may as disclosed be large enough to allow at least two fixing points. The first side bar 2 may be mounted to the bridging element 4 by rivets, screws, adhesive or the like. In the disclosed embodiment screws are used.

By the first and the second flanges 28, 29 being essentially parallel and separated by a distance corresponding to the total thickness T6 of the first side bar 2 and the flange 10 of the first distance element 9, and by the inner surface 32 of the bridging portion 30 being complementary to and in contact with the outer surface 13 of the shoulder 11 of the first distance element 9 there will be essentially no play there between.

The second side bar 3 is rotatably mounted to the bridging element 4 by the bridging element 4 straddling the first end 21 of the second side bar 3 and the flange 23 of the second distance element 22. The flanges 28, 29 of the bridging element 4 are arranged to overlap a portion of the long side 18 of the second side bar 3.

The rotatably mounting of the second side bar 3 to the bridging element 4 may be provided by a hinge pin 33 extending perpendicular to the long sides 18 of the second side bar 3. The hinge pin 33 may be constituted by a screw or the like.

By the flanges 28, 29 of the bridging element 4 being essentially parallel and separated by a distance corresponding to the total thickness T6 of the second side bar 3 and the flange 23 of the second distance element 22, and by the inner surface 32 of the bridging portion 30 being complementary to and in contact with the outer surface 26 of the shoulder 24 of the second distance element 22 there will be essentially no play there between.

In the disclosed embodiment, see FIGS. 2a and 4, the lower corners of the first end 21 of the second side bar 3 and the second distance element 22 respectively facing away from the bridging portion 30 of the bridging element 4 are provided with a radius R. By the radius R, no corner portion will extend exterior of the flanges 28, 29 of the bridging element 4 during rotation of the second side bar 3, see FIG. 2b. Thereby there is no risk of any interference with any clothing of the user.

A locking cavity 35 is defined by the inner walls of the bridging element 4, i.e. the two flanges 28, 29 and the bridging portion 30, together with the two sliding surfaces 27a, 27b, see FIG. 2a.

The locking cavity 35 houses a locking block 36. The locking block 36 has a bar-shaped geometry with a width corresponding to the distance between the inner walls of the two flanges 28, 29 of the bridging element 4. The top surface 37 of the locking block 36 facing the bridging portion 30 of the bridging element 4 is provided with a profile complementary to the profile of the inner surface 32 of the bridging portion 30. The lower surface 38 of the locking block 36 facing the sliding surfaces 27a, 27b of the first and the second distance elements 9, 22 is plane. Also the longitudinal side walls 39 of the locking block 36 are plane.

The locking block 36 is formed as a wedge along its longitudinal extension with its thickest end facing the shoulder 11 of the first distance element 9. The wedge angle α is preferably 1-3 degrees and more preferred 1.5-2.5 degrees, see FIG. 2a. The sliding surface 27b provided by the second side bar 3 is preferably slanted with a complementary angle.

The locking block 36 is in its end facing away from the locking cavity 35 connected to a wire 40 extending through the through channel 15 of the shoulder 11 of the first distance element 9. A biasing member 41, such as a spring is arranged between the shoulder 11 of the first distance element 9 and the locking block 36.

The locking block 36 is movable along and inside the locking cavity 35 between a locked position and an unlocked position by the wire 40 being pulled by the user. The locking block 36 is preferably biased by the biasing member 41 towards the locking position.

In the locked position, see FIG. 2a, the locking block 36 is in wedging engagement with the sliding surface 27b. In this locked position the second side bar 3 is prevented from rotation in view of the bridging element 4. In this position, the locking block 36 is in contact with the sliding surfaces 27a, 27b of both the first and the second side bars 2, 3 by having a length bridging the two sliding surfaces 27a, 27b. Further, the locking block 36 is in contact, preferably without any play, with the inner walls of the flanges 28, 29 and the bridging portion 30 of the bridging element 4.

In the unlocked position, see FIG. 2b, the locking block 36 is pulled towards the first side bar 2, releasing the wedging engagement while being guided by the sliding surface 27a of the first side bar 2, the inner walls of the flanges 28, 29 and the bridging portion 30 of the bridging element 4. The locking block 36 is retracted to a position in which the second side bar 3 is free to rotate in view of the bridging element 4. It is to be understood that the side bar 3 is rotated by the user bending the knee.

As noted, while moving the locking block 36, no matter if it is towards the locking position or the unlocking position, the locking block 36 is guided by being in contact with four longitudinal contact surfaces allowing a very stable guiding and locking.

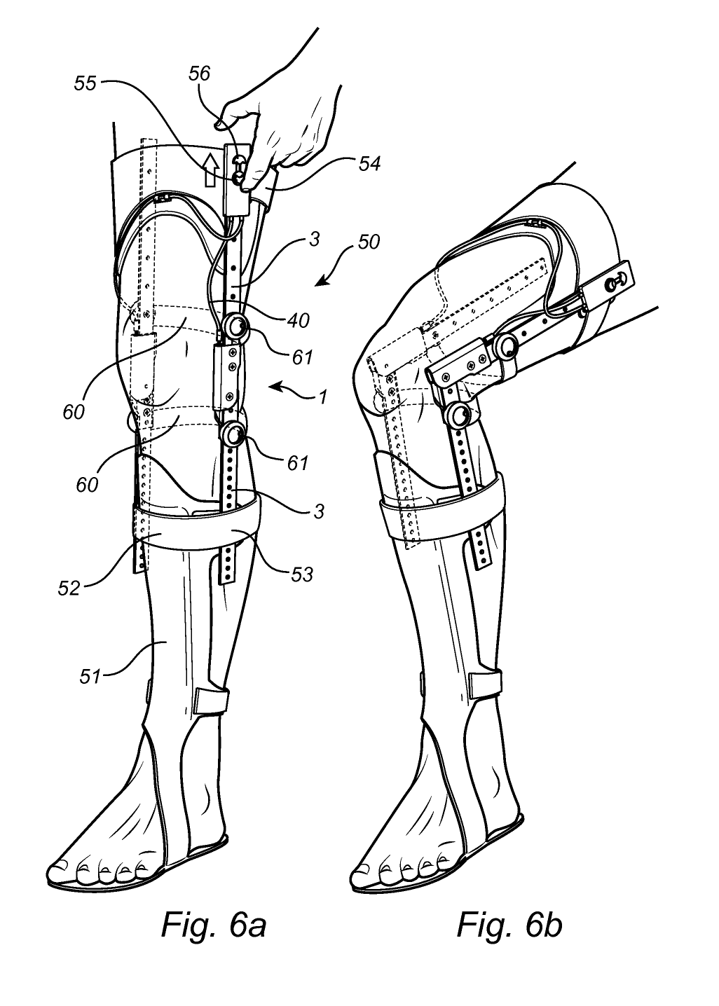

Now turning to FIGS. 6a and 6b, the lockable hinge 1 is disclosed as applied to one example of a knee ankle foot orthosis 50 (KAFO). The KAFO 50 comprises a lower support 51 extending from the lower side of the foot along the lower leg. The lower support 51 is, at its upper end 52 connected to a lower attachment 53 arranged around the leg in a position below the knee. An upper attachment 54 is arranged around the leg in a position above the knee, in the disclosed embodiment around the thigh. The lockable hinge 1 is connected via the free ends of the first and second side bars 2, 3 to the upper and lower attachments 53, 54.

In the disclosed embodiment, the KAFO 50 is further attached to the leg of the user by popliteal straps 60 interconnecting and mounting the medial and lateral locking hinges 1 of the KAFO in parallel to each other. The popliteal straps 60 are attached to optional buckles 61 mounted to the first and second side bars 2, 3.

In the disclosed embodiment, the lockable hinge 1 is arranged on the lateral side of the knee while a similar lockable hinge is arranged on the medial side of the knee. It is to be understood that the lockable hinge on the medial side may be of any other type, such as a non-lockable hinge. The wire 40 used for operation of the locking block is arranged to extend along the thigh towards the upper attachment 54 where it is connected to a remote control 55.

The remote control 55 may comprise a locking mechanism which is remotely connected with the locking block 36 via the wire 40. In the disclosed embodiment the remote control 55 comprises a button 56 which is movable between and lockable in two positions—a first position in which the wire 40 is pulled, thereby releasing the wedging engagement between the locking block 36 and the first side bar 2, whereby the user is free to bend the knee, see FIG. 2b, and a second position in which the tension of the wire 40 is released to such extent that the locking block 36 by the biasing provided by the biasing member 41 returns to its locked position in which the locking block wedgingly engages the first side bar 2 preventing the knee to bend, see FIG. 2a.

It goes without saying the design of the KAFO 50 with its support 51, upper and lower attachments 53, 54 and its tibia connectors 60 may be made in numerous ways and that the invention should not be restricted to the disclosed embodiment. The remote control 55 may by way of example be fastened to the users clothing or to the users leg by a flexible attachment, such as a strap or the like.

Although the inner surfaces of the locking cavity 35 have been described and illustrated as essentially plane with the exception of the guiding portion 30 being curved, it is to be understood that other geometries are possible with the remained function as long as a longitudinal and stable guiding of the locking block 36 may be provided.

It is to be understood that the lockable hinge is applicably also to other body hinges such as the elbow.

The lockable hinge is also applicable to an flexible tube shaped orthosis. The free ends of the side bars may by way of example be received in pockets arranged in the flexible tube.

As a short summary, by the bridging element 4, the locking mechanism comprising e.g. the locking block 36, the sliding surfaces 27a, 27b and the biasing member, is prevented from getting contaminated with fibers from the surroundings. Also, the hinge pin 33 interconnecting the second side bar 3 to the bridging element 4 is protected. Further, all movable parts are encapsulated whereby there is no risk of neither the user nor any surrounding elements, such as clothings or blankets getting stuck in the lockable hinge 1.

No matter position, the locking block 36 is fully guided by the sliding surfaces 27a, 27b and the interior walls 28, 29, 32 of the bridging element 4, whereby the user will experience a very rigid and stable lockable hinge without any play. This is very important or the users comfort. Also, by the wedging action, the rigidity will remain even after being worn. The lockable hinge may be set from the locked to the unlocked position simply by pulling the wire 40. Thereby an easy and simple adjustment is provided requiring no tooling. As disclosed the lockable hinge may even be operated by a remote control.

Further, the lockable hinge has, by the biasing member 41 a built in safety, i.e. if the operation means of the lockable hinge fails, the hinge will go into its locking position. Again this is very important for the users comfort and experienced safety.

Additionally, the lockable hinge is not restricted to being used to the knee, but may be applied also to e.g. the elbow.

Additionally, variations to the disclosed embodiments can be understood and effected by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

1. A lockable hinge for an orthosis, the lockable hinge comprising a first side bar and a second side bar, a bridging element and a locking mechanism, wherein
a first end of the first side bar is fixedly mounted to a first portion of the bridging element, and a first end of the second side bar is rotatably mounted to a second portion of the bridging element, and
the locking mechanism is supported by the first side bar, and the locking mechanism comprises a locking block being slidable along the longitudinal extension of the first side bar between a locked position and an unlocked position, in which locked position the locking block by wedging engages between the second side bar and the bridging element, whereby the second side bar in the locked position is fixedly aligned with the first side bar.

2. A lockable hinge for an orthosis according to claim 1, wherein the bridging element comprises
a first flange overlapping a portion of the long side of the first end of the first side bar and a portion of the long side of the first end of the second side bar; and
a bridging portion overlapping at least a portion of the short side of the first end of the first side bar and a portion of the short side of the first end of the second side bar, the bridging portion extending essentially perpendicular to the first flange, whereby
the locking block is arranged to engage by wedging between the second side bar and an inner surface of the bridging portion.

3. A lockable hinge for an orthosis according to claim 2, wherein the bridging element further comprises
a second flange, opposite the first flange and connected thereto via the bridging portion, whereby
the second flange is overlapping a portion of the opposite long side of the first end of the first side bar and a portion of the opposite long side of the first end of the second side bar.

4. A lockable hinge for an orthosis according to claim 2, wherein
the first and/or the second flanges are arranged to extend essentially in parallel with the long sides of the first and the second side bars, whereas
the bridging portion is arranged to extend essentially in parallel with the short sides of the first side bar and the second side bar.

5. A lockable hinge for an orthosis according to claim 1, further comprising a first distance element comprising a flange and a shoulder, the shoulder extending along the flange, wherein
the flange overlaps a portion of the long side of the first end of the first side bar, and
the shoulder having
an inner surface overlapping a portion of the short side of the first end of the first side bar, and
an outer surface complementary to and in contact with an inner surface of the bridging portion of the bridging element.

6. A lockable hinge for an orthosis according to claim 5, in which the first end of the first side bar is fixedly mounted to the bridging element via the flange of the first distance element and the first and/or the second flange of the bridging element.

7. A lockable hinge for an orthosis according to claim 3, wherein the thickness of the first side bar and the thickness of the flange of the first distance element corresponds to the distance between the first and the second opposing flanges of the bridging element.

8. A lockable hinge for an orthosis according to claim 5, wherein the shoulder comprises a through channel extending along the longitudinal extension of the shoulder, and wherein the through channel receives a wire which in one end is connected to the locking block, whereby the sliding movement of the locking block is operated by the wire being pulled.

9. A lockable hinge for an orthosis according to claim 2, wherein the locking block has an upper guiding surface complementary to the inner surface of the bridging portion of the bridging element.

10. A lockable hinge for an orthosis according to claim 2, wherein the locking block during sliding from the locked position to the unlocked position is guided by the inner surface of the guiding portion of the bridging element and at least one of the first and the second flanges of the bridging element.

11. A lockable hinge for an orthosis according to claim 1, wherein the locking block is biased towards the locked position.

12. A lockable hinge for an orthosis according to claim 1, wherein the locking block is provided with a wedge angle of 1-3 degrees and more preferred 1.5-2.5 degrees.

13. A lockable hinge for an orthosis according to claim 1, wherein the second ends of the first and second side bars are arranged to be received in pockets provided in a flexible knee orthosis.

14. A lockable hinge for an orthosis according to claim 1, wherein the locking block has a longitudinal extension bridging the first ends of the first and the second side bars.

15. A lockable hinge for an orthosis according to claim 2, wherein the locking block is slidably received in a locking cavity defined by the inner wall of the bridging portion and at least one of the flanges of the bridging element and the short sides of the first ends of the first and second side bars.

16. A lockable hinge according to claim 1, wherein the locking lock is operable via a remote control.

17. A lockable hinge according to claim 1, wherein the first and second side bars, the first and second distance elements and the bridging element are made of metal, plastics, composites or a combination of these materials.

18. A knee ankle foot orthosis, a knee orthosis or an elbow orthosis comprising a lockable hinge according to claim 1.

19. A lockable hinge for an orthosis according to claim 3, wherein
the first and/or the second flanges are arranged to extend essentially in parallel with the long sides of the first and the second side bars, whereas
the bridging portion is arranged to extend essentially in parallel with the short sides of the first side bar and the second side bar.
